(19) United States
(12) Patent Application Publication
(10) Pub. No.: US 2013/0317554 A1
(43) Pub. Date: Nov. 28, 2013

(54) LOCKING MECHANISM FOR AN IMPLANTABLE MEDICAL DEVICE

(71) Applicant: Thomas Purcell, Del Mar, CA (US)

(72) Inventor: Thomas Purcell, Del Mar, CA (US)

(21) Appl. No.: 13/478,577

(22) Filed: May 23, 2012

(51) Int. Cl. A61B 17/86

(52) U.S. Cl. 606/319

(57) ABSTRACT

In an implantable medical device, a locking mechanism with enhanced frictional stabilization comprises a substantially spherical elongating spring adapted to expand from a collapsed state to an elongated state, wherein the spring volume is increased in the elongated state for increasing a frictional contact with a surrounding surface. A screw or other protruding element is inserted into the spherical elongating spring for driving the expansion thereof. Locking methods include the expansion of a spring within an aperture of a device body for increasing frictional stabilization therein.

### BACKGROUND OF THE INVENTION

1. Field of the Invention

This invention relates to a locking mechanism and related methods for use with implantable medical devices; and more particularly, to a bushing and screw assembly for mechanically stabilizing an implantable medical device in a fixed state.

2. Description of the Related Art

Implantable medical devices such as bone plates and other implantable devices are well known and used in the art. For several types of medical devices it is often required to provide a locking mechanism, such as a bushing and screw for securing the device to bone or other surface.

Early designs provided plates and other devices with apertures for receiving screws adapted to penetrate bone and secure the implantable device. Initial versions provided screws adapted to extend through the aperture of the plate but not mate therewith. In these early designs, such rudimentary locking mechanisms provided conical articulation and placement of the screws along a preferred axis, however the screws would often tend to move subsequent to the surgery causing discomfort, pain and ultimately disrupting the healing process. In later versions, the apertures of the plate were threaded and adapted to receive a matching thread of the screw in an effort to reduce post-surgical migration of the medical device; however, these later versions provided a limited trajectory of 90 degrees with respect to the plate.

Implantable devices were eventually designed for specific anatomical applications having apertures positioned to receive screws at a predetermined trajectory and locked by matching threads on both the aperture of the plate and the screw. With these anatomically designed locking mechanisms, screw trajectories provided a generally correct anatomical placement; however, the screw trajectory was fixed by the manufacturer of the device and generally failed to provide for variation in anatomy or intra-operative adjustment.

More recently, a longstanding need in the art has driven demand for locking mechanisms adapted to address the needs for: improved conical adjustment of the screw trajectory and setting of the screws by the surgeon prior to or during surgery; improved fixation of the device at the implant site and prevention of device migration; and improved assembly and delivery to the patient.

Presently, a number of proposed solutions have been introduced in the art. For example, certain locking mechanisms provide a variable-angle constrained screw wherein an aperture of a bone plate or other device comprises a threading adapted to receive a matched threading of an inserted screw with the screw capable of engaging the threaded portion of the aperture at an orthogonal trajectory or at up to 30 degrees of conical rotation therefrom. Although these locking mechanisms with variable-angle constrained screws provide additional freedom to a surgeon for assembly and delivery, there is a need to improve stability of these locking mechanisms for preventing migration of the device or other implant malfunctions.

Thus, there remains a need in the art for a locking mechanism for use with implantable medical devices, wherein the locking mechanism is adapted to solve at least the aforementioned problems and limitations in the art. Other needs in the art include improved assembly and setting of the locking mechanism during surgery and low cost manufacturing of these locking mechanisms.

### SUMMARY OF THE INVENTION

An improved locking mechanism is provided for stabilizing an implantable medical device in a fixed state. The locking mechanism comprises a bushing and screw assembly compatible with implantable bone plates and a variety of other implantable medical devices.

The locking mechanism generally comprises at least a portion of a medical device having an aperture extending along a through-hole axis from a first side to a second side opposite of the first side. The aperture further comprises a substantially spherical inner surface having a pair of opposing planar walls oriented substantially parallel with one another and aligned in the direction of the through-hole axis. In addition to the aperture, the locking mechanism further comprises a spherical elongating spring, and a screw. The spherical elongating spring comprises a substantially spherical outer surface thereof having a pair of opposing planar surfaces oriented substantially parallel with one another and aligned in the direction of a spring axis. The spherical elongating spring is adapted to expand or elongate along the spring axis from a collapsed state to an elongated state upon engagement with a threaded portion of the screw, thereby elongating a corresponding outer surface area and volume of the spherical elongating spring to engage an inner contact patch of the inner aperture cavity for providing improved frictional stabilization.

In this regard, the spherical elongating spring in a collapsed state is inserted into an aperture of the device with the spring axis being perpendicular to the through-hole axis and such that the opposing planar surfaces of the spring are engaged with the opposing planar walls of the aperture. Subsequent to insertion, the spherical elongating spring is rotated about the opposing planar surfaces until the spring axis substantially aligns with the through-hole axis or within up to 30 degrees of conical rotation therewith. The screw is then inserted through the spring along the spring axis and at least one engagement member of the screw is engaged with a corresponding elongation element of the spherical elongating spring, resulting in the expansion of the spring from the collapsed state to an elongated state, wherein the locking mechanism is secured with added frictional stability with the spring in the elongated state.

In one embodiment, a spring busing is elongated within an aperture or cavity of an implantable medical device upon rotation of an inserted screw. In this regard, the elongated spring forms a frictional engagement with a surrounding surface to provide a locking therebetween.

In certain embodiments, the locking mechanism provides an improvement with regard to ease of assembly wherein the locking mechanism is comprised of three parts; i.e. an aperture extending through the device, a spherical elongating spring, and a screw. Additionally, the limited parts provide a corresponding low-cost enhancement over prior art locking mechanisms for implantable devices.

Other features and improvements will become apparent to those having skill in the art upon further review of the detailed description, and in particular when reviewed in conjunction with the appended figures.

### BRIEF DESCRIPTION OF THE DRAWINGS

FIG. 1 is a perspective view of an implantable medical device comprising a number of locking mechanisms in accordance with embodiments herein.

FIGS. 2(a-b) illustrate perspective views of an implantable medical device comprising a plurality of apertures in accordance with the embodiment of FIG. 1, and a through-hole axis with respect to at least one aperture, respectively.

FIG. 3 is a perspective view of a screw in accordance with embodiments herein, the screw comprises a first thread portion extending along a body of the screw and a second threaded portion extending along a head of the screw.

FIGS. 4(a-b) illustrate perspective views of a spherical elongating spring comprising one or more slots extending radially outward from a center of the spring in a direction perpendicular to a spring axis, and a pair of planar opposing walls oriented in the direction of the spring axis, respectively.

FIG. 5a is a side view of a portion of an implantable device comprising an aperture and inserted spherical elongating spring in accordance with the embodiments herein.

FIG. 5b is a side view of a plurality of apertures and spherical elongating springs disposed within an implantable device.

FIGS. 6(a-c) illustrate several views depicting assembly of a locking mechanism within an aperture of an implantable medical device.

FIG. 7 is a side view of the locking mechanism illustrating an engagement between a threaded portion of a screw and a threaded portion of a spherical elongating spring within an aperture of an implantable medical device.

FIG. 8 illustrates a side view of a locking mechanism and the action of elongatingelongating a spherical elongating spring within an aperture of the implantable device.

FIG. 9 illustrates a spherical elongating spring in an elongated state wherein the spring is elongated along the spring axis.

### DETAILED DESCRIPTION OF THE PREFERRED EMBODIMENTS

In the following description, for purposes of explanation and not limitation, details and descriptions are set forth in order to provide a thorough understanding of the present invention. However, it will be apparent to those skilled in the art that the present invention may be practiced in other embodiments that depart from these details and descriptions.

Although the locking mechanism will be hereinafter described according to embodiments relating to bone plates, it should be understood that the various embodiments can be incorporated into other implantable medical devices with little variation or deviation from the embodiments disclosed herein. Accordingly, the scope of the invention is not intended to be limited to the illustrated embodiments, but rather shall be set forth in the appended claims.

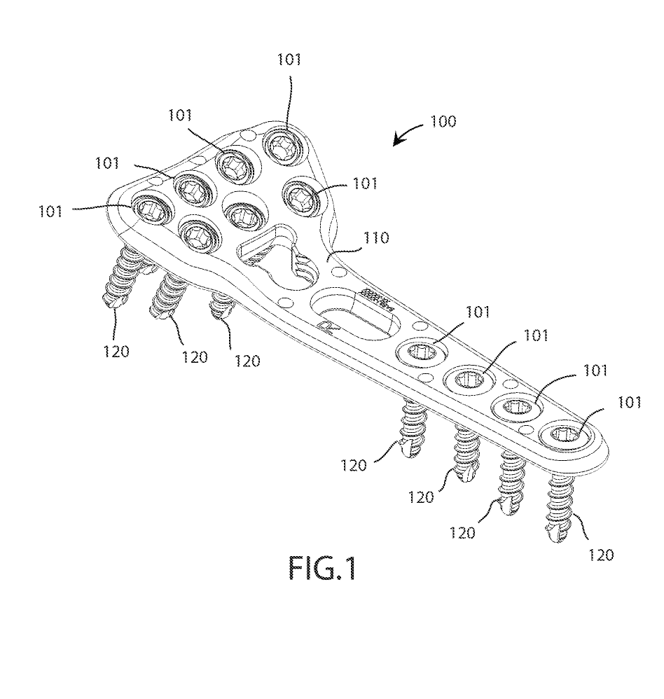

Now turning to the drawings, FIG.1 illustrates an implantable medical device 100 and a plurality of locking mechanisms 101 according to embodiments herein. The medical device comprises a bone plate having a plurality of apertures extending from a top surface 110 to a bottom surface of the implantable device. A busing and screw 120 are individually inserted into one or more of the apertures of the device to yield the locking mechanisms 101.

It should be noted that in the illustrated embodiments it is advantageous to insert one or more screws into a portion of the bone for promoting healing thereabout; however the locking mechanisms described herein can further be used to attach two or more components or attach other suitable medical devices within a targeted delivery site.

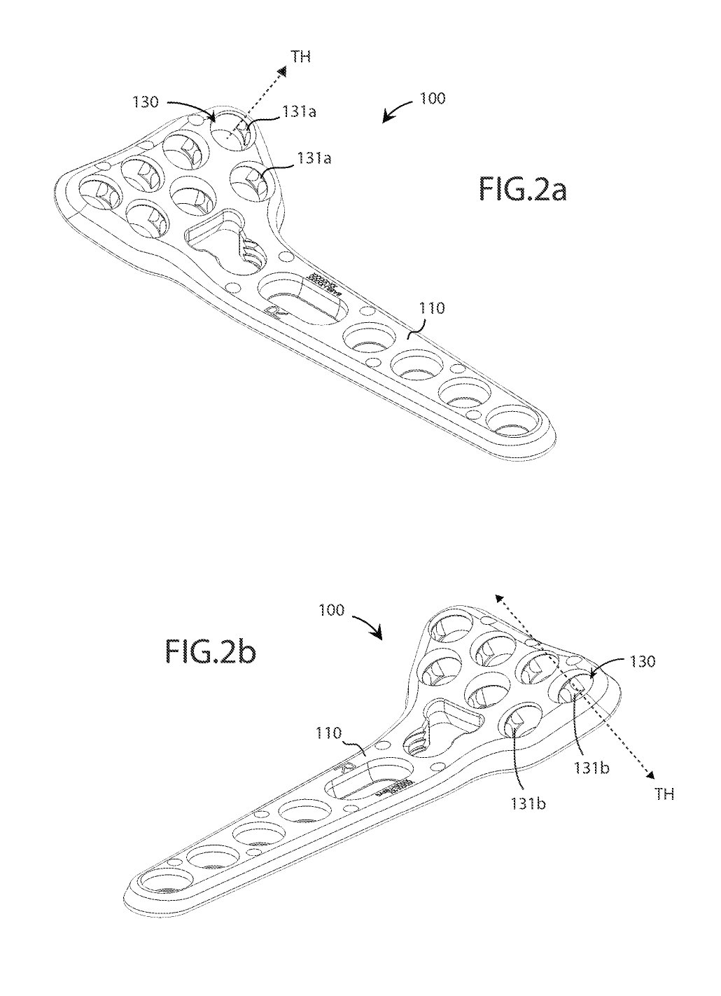

FIGS. 2(a-b) illustrate an implantable medical device from alternative perspectives, wherein the implantable device 100 comprises at least one aperture 130 having a substantially spherical inner surface thereof and pair of planar opposing walls 131a; 131b. The aperture 130 extends through a portion of the implantable device from a bottom surface to a top surface 110 along the through-hole axis (TH). Each of the planar opposing walls 131a; 131b of the aperture 130 is oriented in a plane that is substantially parallel with respect to the through-hole axis (TH).

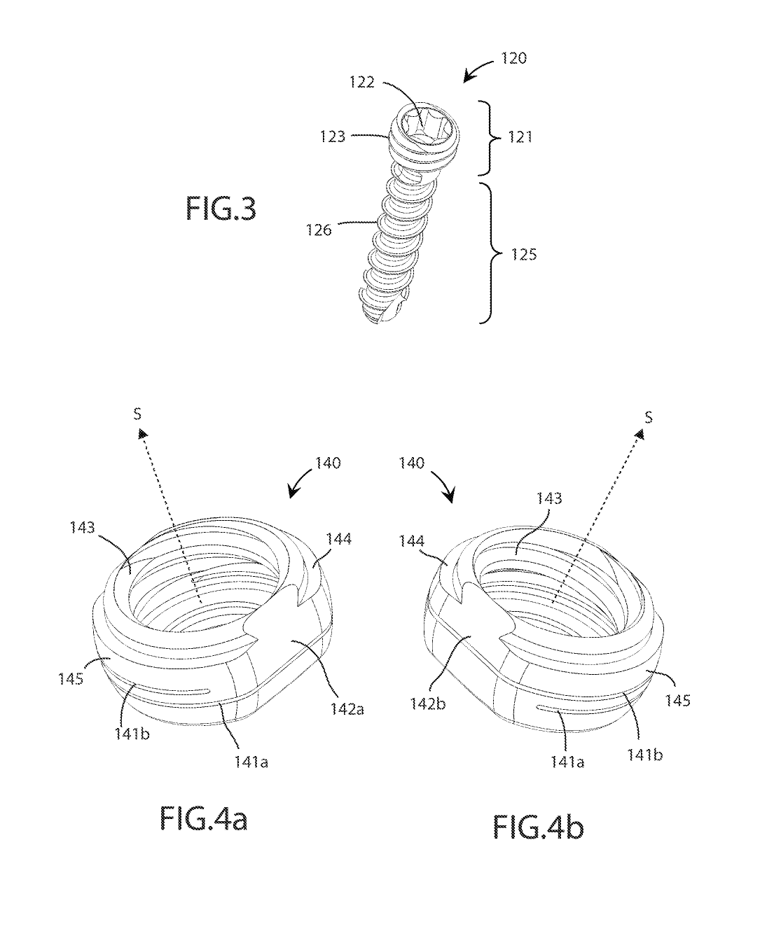

FIG. 3 illustrates a screw for use in a locking mechanism according to embodiments herein. The screw 120 comprises a body portion 125 and a head portion 121. The body portion 125 further comprises a first threaded portion 126 extending along a body of the screw from the head portion to a distal tip. The head portion 121 further comprises a second threaded portion 123 extending from the body portion to a proximal rim of the screw head. At the head of the screw is disposed an engagement cavity 122 adapted to receive a tool for rotational translation. Various screw engagement cavities can be used including hexagonal, straight, Phillips, cruciform, and torx, however it may be suggested to use a screw having a torx or hexalobe engagement cavity as these screws are well known for efficiency and durability with torque applications.

The screw, or more broadly the “protruding element”, may comprise one or more continuous threaded portions as is common within the art. Alternatively, the screw may comprise one or more discontinuous engagement portions. Accordingly, for purposes of this invention the term “engagement members” is provided to collectively include continuous threaded portions, discontinuous threaded portions, helical cams and wedges and any other engagement or gripping type surface adapted for rotational engagement. In this regard, the protruding element may comprise one or more engagement members disposed thereon.

Of particular importance, those having skill in the art will recognize that the “screw” as used herein is not intended to be limiting to traditional models. In certain embodiments, a pin or elongated rod may comprise one or more engagement members adapted to engage an elongation portion of the spring. Accordingly, the term “screw” is intended to be broadly construed and includes any elongated structure adapted to engage an elongation portion of a corresponding spherical elongating spring for the purpose of elongating the spring volume in the direction of the spring axis. The term “protruding element” may be used herein to describe a general structure for effectuating elongation of the spherical elongating spring, including a screw, pin, elongated rod, or other structure adapted to elongate the spring in a direction of the spring axis.

FIGS. 4(a-b) illustrate a spherical elongating spring 140 from alternative perspectives in accordance with various embodiments of the locking mechanism. The spherical elongating spring 140 comprises a substantially spherical outer surface 145 and an inner spring surface adapted to engage a portion of a screw. The spherical elongating spring extends along the spring axis (S) from a bottom edge to a top edge thereof. A pair of planar opposing surfaces 142a; 142b extend along the outer spring surface at opposite sides of the spring body. The planar opposing surfaces are oriented parallel with respect to each other and the spring axis (S). One or more slots 141a; 141b extend radially outwardly from a center of the spring body in a direction perpendicular to the spring-axis (S). The inner volume of the spring is hollowed out to form a lumen extending therethrough along the spring axis (S). At least a portion the lumen or cavity of the spring comprises one or more elongation elements 143 collectively defining an elongation portion thereof. The elongation portion can be a threaded portion at least partially extending along the inner surface of the spring. Alternatively, the elongation portion may comprise one or more helical cams or helical wedges in a discontinuous pattern wherein the elongation portion is adapted to receive an engagement member of a screw for elongating the spring from a collapsed state to an elongated state. Optionally, the spherical elongating spring may comprise a circumferential shelf 144 extending along a circumference of the spring at one or more of a top and bottom edge thereof.

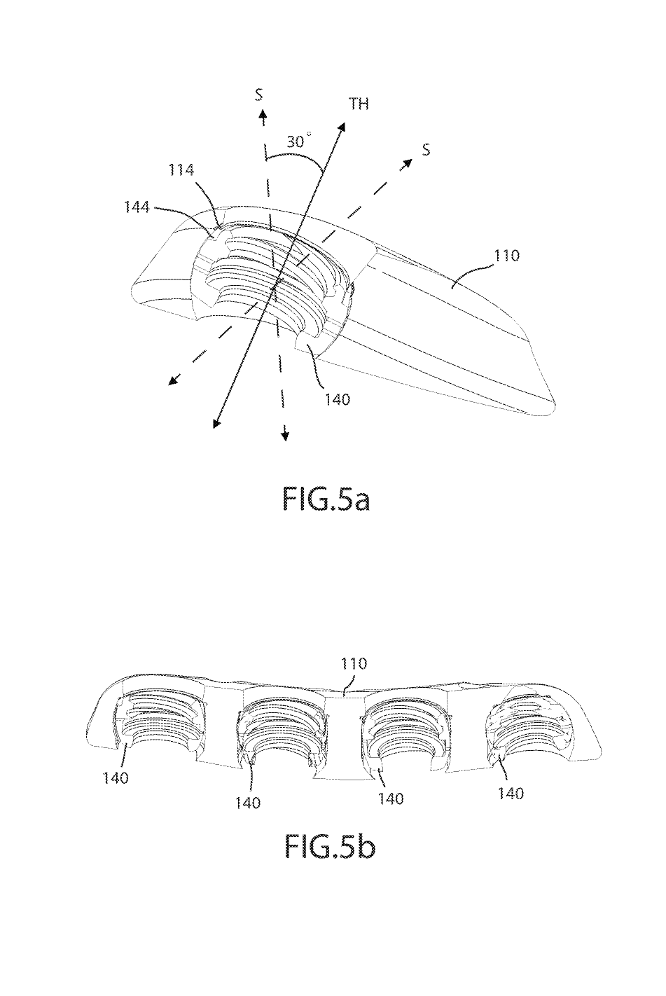

FIG. 5a is a sectional view illustrating a portion of an implantable medical device 110 comprising an aperture and a spherical elongating spring 140 disposed therein. The aperture defines a through-hole axis (TH) whereas the spring defines a spring axis (S). FIG. 5a further illustrates the circumferential shelf 144 of the spherical elongating spring and a circumferential rim 114 of the aperture adapted to restrict movement of the spherical elongating spring 140 such that the spring axis (S) is confined within a thirty degree)(30° conical rotation about the through-hole axis (TH) of the aperture.

FIG. 5b illustrates a portion of an implantable medical device 110 comprising multiple apertures and springs 140 disposed therein. In this regard, an implantable medical device may incorporate one or more locking mechanisms in accordance with the embodiments herein for the purpose of providing a secure fitment between device components, or between a device and a targeted delivery site in situ.

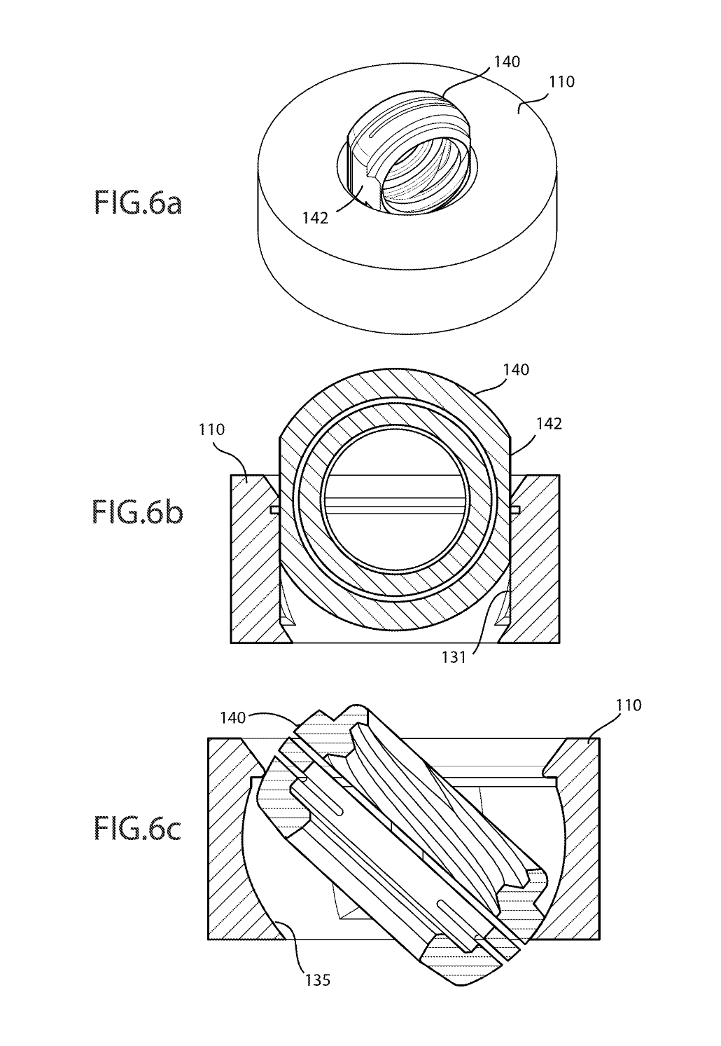

FIG. 6(a-c) illustrate multiple consecutive views illustrating chronological assembly of a locking mechanism in accordance with one embodiment.

FIG. 6a is a perspective view of a portion of an implantable medical device 110 comprising an aperture having a substantially spherical inner surface and a pair of planar opposing walls extending vertically along a through-hole axis as described above. The spherical elongating spring 140 is in a collapsed state at rest and further comprises a pair of planar opposing surfaces 142. The spherical elongating spring is inserted into the aperture with the spring axis perpendicular to the through-hole axis and the planar opposing surfaces of the spring aligned parallel with the planar opposing walls of the aperture.

FIG. 6b is a side view of the spring being inserted into the aperture as illustrated in FIG. 6a and described above. As illustrated, a portion of an implantable medical device 110 comprises an aperture substantially having an inner spherical surface with exception of a pair of planar opposing walls 131. The spring 140 is oriented such that the planar opposing surfaces 142 thereof are aligned with the planar walls of the aperture, and the spring is inserted into the aperture.

FIG. 6c is a side view of the spring disposed within the aperture. The spherical elongating spring 140 is rotated once fully inserted within the aperture such that the spring axis becomes aligned within thirty degrees (30°) conical rotation of the through-hole axis. As illustrated, a portion of the inner surface of the aperture that is adjacent to the planar walls 131 comprises a substantially spherical inner surface 135.

Once inserted, the spring is adapted to receive a portion of an inserted screw. As described above, a plurality of screw types may be used, however the illustrated embodiment depicts a torx screw having a body portion having a first threading portion thereon and a head portion having a second threading portion.

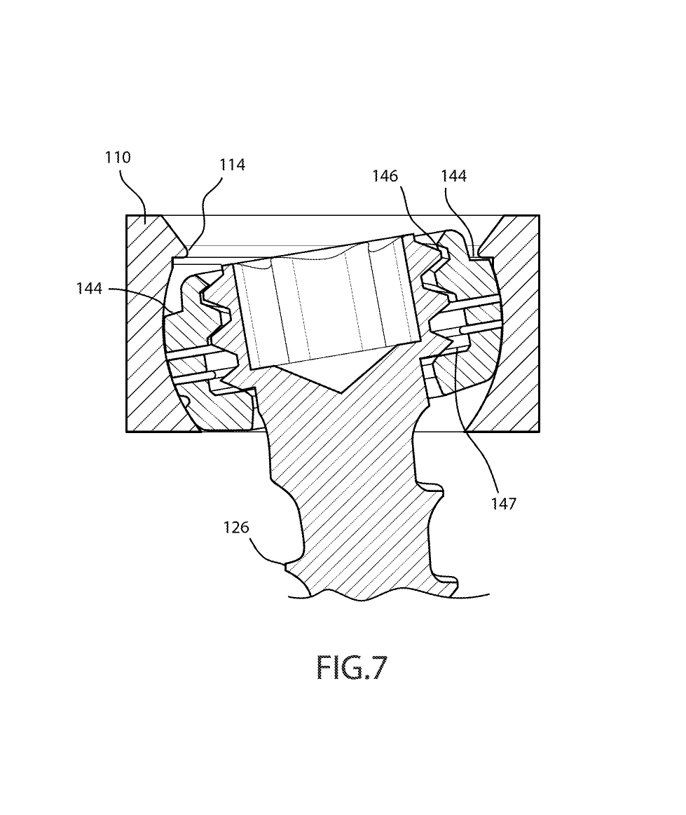

FIG. 7 illustrates a side view of the locking mechanism comprising a portion of an implantable medical device 110 having an aperture extending therethrough, a spherical elongating spring disposed within the aperture, and a screw inserted within the spherical elongating spring. From this side view it becomes clear that the spherical elongating spring provides a configurable busing adapted to expand a volume thereof upon insertion and engagement with a screw. As illustrated, the screw comprises engagement members adapted to engage the elongation portion of the spring for elongating said spherical elongating spring from a collapsed state to an elongated state. The aperture further comprises a circumferential rim 114 adapted to restrict movement of the inserted spring upon contact with the circumferential shelf 144 of the spring. The engagement members of the screw disposed at the head portion thereof are rotated as the screw is inserted through the spring lumen such that the engagement members rotationally engage the elongation elements of the spring.

Moreover, it will be recognized that a multitude of elongation elements and designs thereof can be incorporated into the spring. For illustration, FIG. 7 depicts a spring having a first elongation element 146 disposed at a top of the inner lumen surface of the spring, and a second elongation element 147 disposed a a bottom of the inner lumen surface of the spring. In this regard, the engagement members of the screw are adapted to rotatably engage both the first and second elongation elements of the spring and ultimately translate the second elongation element outwardly from the first elongation element resulting in an elongated spring.

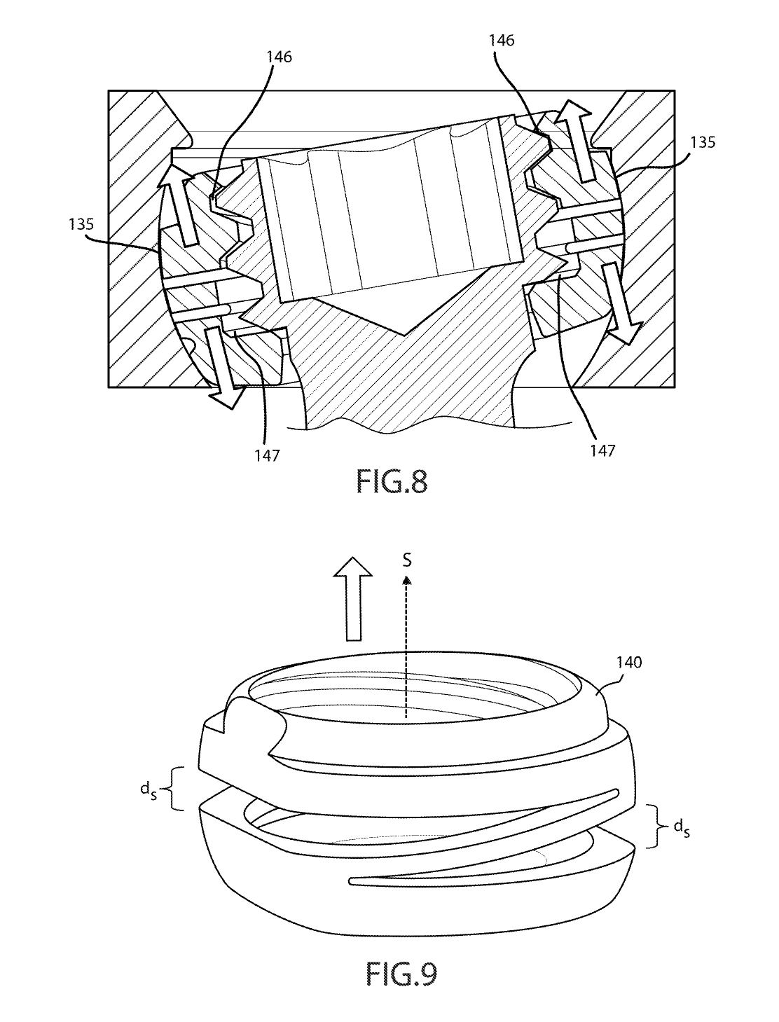

FIG. 8 illustrates the embodiment of FIG. 7 wherein the spring is elongated from a collapsed state to an elongated state upon rotational adjustment of the screw within the spherical elongating spring. As illustrated, a first elongation element 146 at a top portion of the spring translates outwardly from a second elongation element 147 disposed at a bottom portion of the spring with consideration of the spring axis, thereby increasing a volume of the spring and thus increasing a contact patch for which the spring becomes securely nested within the inner spherical surface 135 of the aperture.

FIG. 9 illustrates the spherical elongating spring in an elongated state as illustrated in FIG. 8 and described above. As illustrated, a gap (dₛ) between the slots is increased as the spring 140 expands along the spring axis (S). Accordingly, an outer surface area contact patch is increased for providing increased stability when the spring is elongated within the aperture as described above.

Accordingly, a locking method according to one embodiment comprises: inserting a spring into an aperture of a medical device; rotating a screw within an inner volume of said spring and engaging one or more engagement members of the screw with elongation elements of the spring; and elongating said spring within said aperture to increase a frictional engagement therebetween. More particularly, the inserting a spring comprises: aligning at least one planar surface of the spring with a planar wall of the aperture; sliding said planar surface of the spring along said planar wall of the aperture to insert said spring into the aperture; and rotating said spring within the aperture to substantially align a spring axis extending through the spring with a through-hole axis extending through the aperture.

Moreover, the spherical elongating spring can be manufactured within a desired tolerance such that the spring is easily inserted within the aperture and yet further provides sufficient friction to promote alignment of the spring axis without an inserted screw. In this regard, the spring can be positioned with the spring axis (S) oriented at a desired trajectory with the spring in the collapsed state. This initial friction is provided by the contact patch formed between the spring in the collapsed state and the inner spherical surface of the aperture. Note that as the screw is inserted and tightened and the spring is elongated, the volume of the spring is increased such that the resulting contact patch between the elongated spring and the surrounding surface of the aperture provides a secure and stabilized frictional fit. This is an improvement over contemporary locking mechanisms since the described locking mechanism provides a first frictional contact patch for providing configuration of the locking mechanism and screw trajectory, as well as a second frictional contact patch for securing the locking mechanism in the desired orientation.

In addition, the above-described locking mechanism is capable of assembly and configuration prior to surgery, thereby reducing surgery duration and improving the quality of the surgery. For example, using various imagery, a surgeon can assemble the implantable device with one or more locking mechanisms prior to the surgery knowing from the imagery certain desired trajectories for inserting screws between implantable tissue or bone. With the implantable device substantially configured for surgery, the surgeon can significantly reduce delivery time and effort with only minor adjustments as needed since the device is capable of pre-surgery configuration.

Because the locking mechanism comprises three essential components, i.e. an aperture, an spherical elongating spring, and a screw, each as described above, the manufacturing and downstream costs are significantly reduced. In addition, assembly time is reduced with the simplified componentry.

Although various embodiments have been illustrated and described herein it should be understood by those having skill in the art that a number of feature variations and alternative configurations may be achieved without departing from the spirit and scope of the invention as set forth in the appended claims.

What is claimed is:

1. A locking mechanism for stabilizing an implantable medical device, comprising:
at least a portion of a device housing comprising an aperture thereon, said aperture extending from a first side to a second side thereof along a through-hole axis, an inner surface of the aperture being substantially spherical and further comprising a pair of opposing planar edges oriented substantially parallel with one another and aligned in a direction of the through-hole axis;
a spherical elongating spring having a first volume in a collapsed state and a second volume in an elongated state,
the spherical elongating spring comprising:
a substantially spherical body with one or more slots extending from an inner surface to an outer surface thereof,
a pair of opposing planar surfaces disposed on the outer surface, the opposing planar surfaces being oriented substantially parallel with one another and aligned in a direction of a spring axis, and
a lumen formed at the inner surface and extending along the spring axis, at least a portion of the lumen comprising one or more elongation elements collectively defining an elongation portion thereof; and
a screw comprising one or more engagement members adapted to engage said elongation portion for elongating said spherical elongating spring from said collapsed state to said elongated state;
wherein said spherical elongating spring is adapted to provide increased frictional stabilization with said inner surface of said aperture in the elongated state.

2. The locking mechanism of claim 1, wherein said aperture is disposed at an insert adapted for insertion within a portion of the device housing.

3. The locking mechanism of claim 1, wherein said aperture further comprises a circumferential rim extending along a circumference of the aperture at one of said first and second sides thereof.

4. The locking mechanism of claim 3, wherein said spherical elongating spring further comprises a circumferential shelf extending along an outer surface circumference of the spherical elongating spring at one of said first and second sides thereof.

5. The locking mechanism of claim 4, wherein a center of said aperture defines an apex.

6. The locking mechanism of claim 5, wherein said screw extends through said apex and is oriented along said through-hole axis.

7. The locking mechanism of claim 5, wherein said screw extends through said apex and is not oriented along said through-hole axis.

8. The locking mechanism of claim 5, wherein said circumferential rim and said circumferential shelf are adapted to restrict an orientation of said screw such that said screw is adapted to extend through said apex at up to thirty degrees of conical rotation about said through-hole axis.

9. The locking mechanism of claim 1, wherein said spherical elongating spring is adapted to provide a first contact patch about said inner aperture surface when configured in the collapsed state.

10. The locking mechanism of claim 9, wherein said spherical elongating spring is adapted to provide a second contact patch about said inner aperture surface when configured in the elongated state, said second contact patch having greater surface area than said first contact patch.

11. The locking mechanism of claim 1, wherein said elongation elements are individually selected from: threads, helical cams, and helical wedges.

12. The locking mechanism of claim 11, wherein said engagement members are individually selected from: threads, helical cams, and helical wedges.

13. The locking mechanism of claim 1, wherein said screw does not comprise threads.

14. In an implantable medical device, a locking mechanism comprising: a spherical elongating spring extending from a first end to a second end along a spring axis, said spherical elongating spring having an inner lumen surface comprising one or more elongation elements adapted to engage one or more engagement members of an inserted protruding element, wherein said spherical elongating spring is adapted to expand along said spring axis from a first collapsed state to a second elongated state for increasing a frictional contact between the spherical elongating spring and a surrounding surface.

15. The locking mechanism of claim 14, wherein said surrounding surface comprises an inner spherical surface of an aperture disposed about the implantable medical device.

16. A locking method for use with an implantable medical device, comprising:
inserting a spring into an aperture of a medical device;
rotating a protruding element within an inner volume of said spring and engaging one or more engagement members of the protruding element with elongation elements of the spring; and
elongating said spring within said aperture to increase a frictional engagement therebetween.

17. The locking method of claim 15, said inserting a spring comprising:
aligning at least one planar surface of the spring with a planar wall of the aperture;
sliding said planar surface of the spring along said planar wall of the aperture to insert said spring into the aperture; and
rotating said spring within the aperture to substantially align a spring axis extending through the spring with a through-hole axis extending through the aperture.
